(19) United States
(12) Patent Application Publication
(10) Pub. No.: US 2009/0292308 A1
(43) Pub. Date: Nov. 26, 2009

(54) SPINAL FIXATION SYSTEM

(75) Inventors: Scott Jones, McMurray, PA (US); Richard W. Woods, Catonsville, MD (US); Andrew Thomas Rock, Spring Grove, PA (US); Michael Barrus, Ashburn, VA (US)

Correspondence Address: CARTER, DELUCA, FARRELL & SCHMIDT, LLP, 445 BROAD HOLLOW ROAD, SUITE 420, MELVILLE, NY, 11747, US

(73) Assignee: K2M, Inc., Leesburg, VA (US)

(21) Appl. No.: 12/470,874

(22) Filed: May 22, 2009

Related U.S. Application Data
(60) Provisional application No. 61/055,246, filed on May 22, 2008.

(51) Int. Cl. A61B 17/86; A61B 17/70; A61B 17/88

(52) U.S. Cl. 606/205; 606/301; 606/264; 606/279

(57) ABSTRACT

A spinal fixation system includes a bone screw assembly, a low profile bone screw assembly, and a spinal rod. The bone screw assembly may include a separate locking structure. A laminar hook may be coupled to the rod. The spinal rod is operatively connected to the bone screw assembly and the low profile bone screw assembly.

### BACKGROUND

1. Technical Field

The present disclosure relates to orthopedic surgery. More particularly, the present disclosure relates to devices for stabilizing and fixing the bones and joints of the spine.

2. Background Art

The spinal column is a complex system of bones and connective tissues that provides support for the human body and protection for the spinal cord and nerves. The adult spine is comprised of 24 vertebral bodies, which are subdivided into three areas including seven (7) cervical vertebrae, twelve (12) thoracic vertebrae and five (5) lumbar vertebrae. Between each vertebral body is an intervertebral disc that cushions and dampens the various translational and rotational forces exerted on the spinal column.

There are various disorders, diseases and types of injury which the spinal column may experience in a lifetime. The problems may include, but are not limited to, scoliosis, kyphosis, excessive lordosis, spondylolisthesis, slipped or ruptured discs, degenerative disc disease, vertebral body fracture, and tumors. Persons suffering from any of the above conditions typically experience extreme or debilitating pain and often times diminished nerve function.

One of the more common solutions to any of the above mentioned conditions involves a surgical procedure known as spinal fusion. A spinal fusion procedure involves fusing two or more vertebral bodies in order to eliminate motion at the intervertebral disc or joint. To achieve this, natural or artificial bone, along with a spacing device, replaces part or all of the intervertebral disc to form a rigid column of bone and mechanical hardware. In this way damaged or diseased vertebrae are connected to healthy adjacent vertebrae to stabilize the spine while the bone grows and fusion takes place.

The mechanical hardware used to immobilize the spinal column typically involves a series of bone screws and metal rods or plates. When the spine surgery is performed anteriorly, it is common practice to attach a thin metal plate directly to the vertebral bodies and secure it to each vertebral level using one or more bone screws. When the spine surgery is posteriorly performed, it is common practice to place bone screws into the vertebral bodies and then connect a metal rod between the bone screws, thereby creating a rigid structure between adjacent vertebral bodies. The remainder of this disclosure will relate to this second method of performing spinal fusion, wherein the vertebral bodies are connected using a series of bone screws connected by a metal rod.

Traditional set screw assemblies include a screw that is implanted into a vertebral body and a locking nut that secures a metal rod to the screw. Traditional set screw assemblies arc disclosed in U.S. Pat. Nos. 6,471,705, 6,296,642, 6,280,442, 6,074,391, 6,053,917, 5,885,286, 5,879,350, 5,797,911, 5,735,851, 5,733,286, 5,733,285, 5,716,356, 5,672,176, 5,549,608, 5,474,555, 5,466,237, and 5,207,678.

Low profile screws, like those described in U.S. Pat. Nos. 6,840,940, 6,827,719, 6,451,021, 5,683,392 and 7,090,674, and U.S. Patent Application Publication No. 2006/0276792 also are known.

### SUMMARY

A spinal fixation assembly includes at least one low profile bone screw assembly, a bone screw assembly, and a spinal rod. The spinal rod is operatively coupled to the bone screw assembly and the low profile bone screw assembly. Each of the disclosed bone screw assemblies are attachable to separate vertebral bodies. A rod and screw construct is formed when the spinal rod is coupled to two of the bone screw assemblies. A laminar hook may be attached to the spinal rod. A locking device may be used with one of the bone screw assemblies for securing the spinal rod with respect to the bone screw assemblies. The bone screw assemblies may be formed from titanium, titanium alloy, or another biocompatible material. The spinal rod may be formed from cobalt chrome, polyetheretherketone (“PEEK”), or another biocompatible material.

A method for using the presently disclosed spinal fixation system includes attaching at least one low profile bone screw assembly to a selected region of a patient's spine and attaching at least one bone screw assembly to a different region of the patient's spine. A spinal rod is attached to the at least one low profile bone screw assembly and the at least one bone screw assembly.

### DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1A is a side view of a spinal fixation system according to an embodiment of the present disclosure;

FIG. 1B is a perspective view of the spinal fixation system of FIG. 1A;

FIG. 2A is a perspective view of the base and coupling element of the set screw assembly of FIGS. 1A and 1B;

FIG. 2B is an enlarged top view of the base and coupling element of FIG. 2A;

FIG. 3A is a perspective view of a locking insert of the set screw assembly of FIGS. 1A and 1B;

FIG. 3B is a side view of the locking insert of FIG. 3A;

FIG. 4A is a perspective view of a low profile screw assembly of FIGS. 1A and 1B;

FIG. 4B is an enlarged top view of the low profile screw assembly of FIG. 4A;

FIG. 5A is top view of a spinal fixation system of the present disclosure affixed to a vertebral column; and

FIG. 5B is a perspective side view of the spinal fixation system of FIG. 5A.

### DETAILED DESCRIPTION

While the present disclosure will be described more fully hereinafter with reference to the accompanying drawings, in which particular embodiments and methods of spinal fixation are shown, it is to be understood at the outset that persons skilled in the art may modify the embodiments herein described while achieving the functions and results of this disclosure. Accordingly, the descriptions which follow are to be understood as illustrative and exemplary of specific structures, aspects and features within the broad scope of the present disclosure and not as limiting of such broad scope.

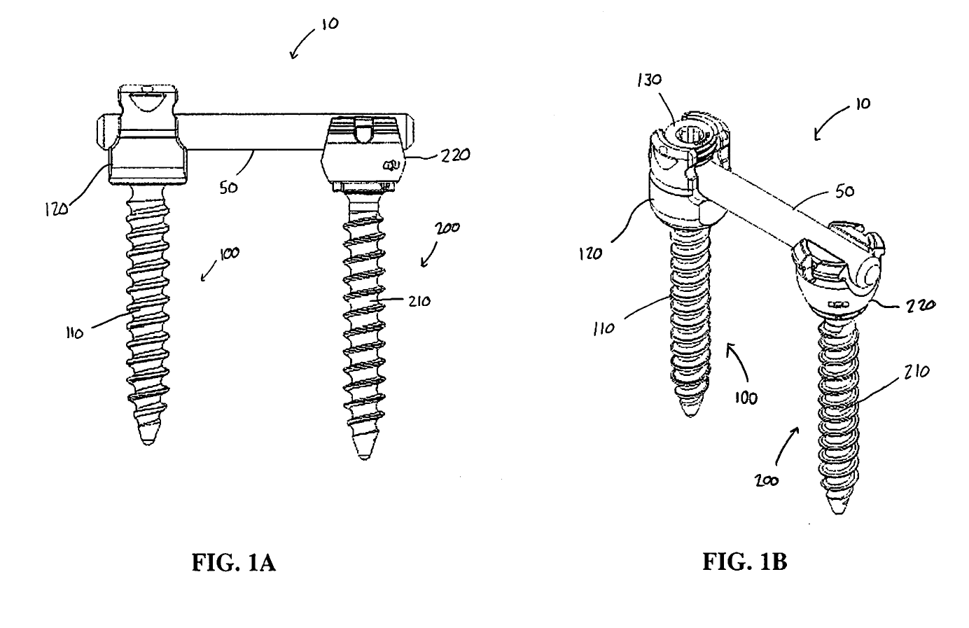

Referring initially to FIGS. 1A and 1B, a spinal fixation system according to aspects of the present disclosure is shown generally as spinal fixation system 10. Spinal fixation system 10 includes at least one set screw assembly 100, a low profile screw assembly 200, and a connector rod 50 extending therebetween. While both set screw assemblies and low profile screw assemblies are known and have been used independently of each other, applicants believe they are the first to propose use of both a low profile design (such as a taper lock screw) in combination with a non-low profile screw such as a set screw or top locking nut design in the same construct. While it is envisioned that the aspects of the present disclosure may incorporate any traditional set screw assembly and any low profile screw assembly, for convenience the following discussion will relate to a low profile taper lock screw assembly 100 and a traditional set screw assembly 200. The aspects of the present disclosure should not be read as limited to the low profile or traditional set screw assemblies 100, 200 described herein.

Briefly, set screw assembly 100 includes a base 110, a coupling element 120 operably connected to base 110, and a locking insert 130 threadingly received within coupling element 120 for selectively securing connector rod 50.

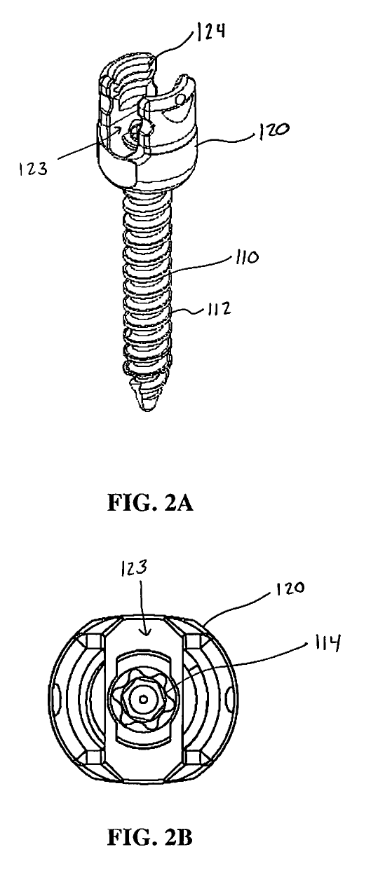

With reference now to FIGS. 2, base 110 of set screw assembly 100 includes threaded shaft 112 configured for insertion into bone, and a curvate head (not shown) defining a slot, opening or recess 114 for receiving a driving device. By way of example only, slot 114 may engage a screw driver or more specifically a hex screw driver (not shown). Pivotably mounted on base 110 is coupling element 120. Coupling element 120 is a substantially tubular member with a rod receiving channel 123 formed in the top thereof. Rod receiving channel 123 includes a threading 124 for receiving locking insert 130. Alternatively, threading 124 may be provided on the outer upper surface of coupling element 120 to engage locking nut or combined nut/screw.

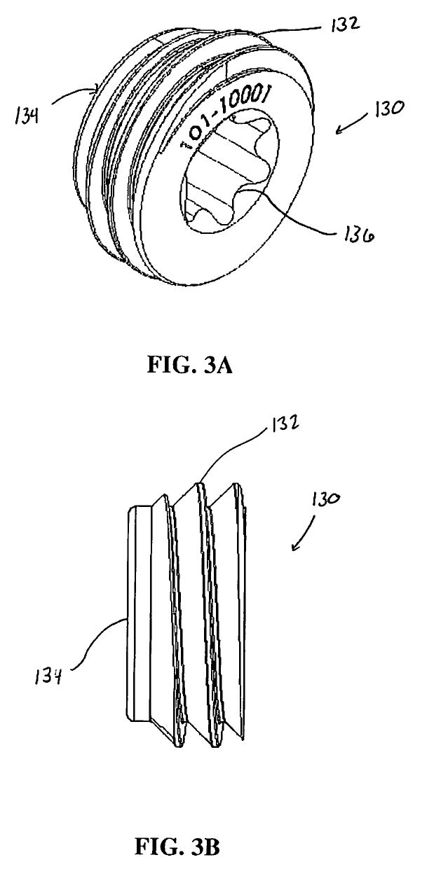

With reference to FIG. 3, locking insert 130 includes a cylindrical body having a threading 132 thereon. Threading 132 is configured to mate with threading 124 formed in coupling member 120. In an alternative embodiment, wherein threading 124 of the coupling element 130 is provided on the exterior surface thereof, locking insert 130 is replaced by a locking nut (not shown). Locking insert 130 further may include a flat, or ridged underside 134 which is ideally suited to gripping a rod surface. The upper portion of locking insert 130 includes a recess 136 into which a screw driving tool may be inserted to drive locking insert 130 into coupling element 120.

For a more detailed description of set screw type assembly 100 and the operation thereof, please refer to commonly owned U.S. Pat. No. 5,733,286, the contents of which are hereby incorporated by reference herein in its entirety. One suitable set screw locking pedicle screw is available under the brand name DENALI® (K2M, Inc., Leesburg Va.).

Referring back to FIGS. 1A and 1B, briefly, low profile screw assembly 200 includes a base 210 and a housing 220 operably connected to base 210 for selectively receiving connector rod 50.

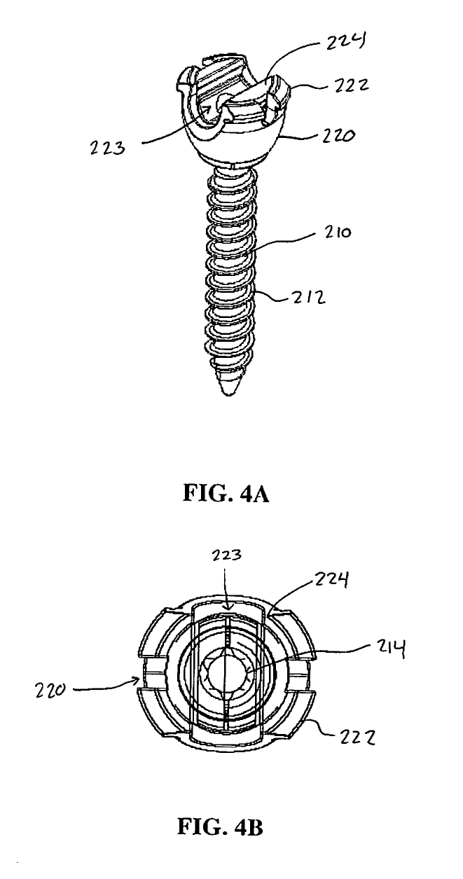

With reference to now to FIGS. 4A and 4B, base 210 of low profile screw assembly 200 includes a threaded shaft 212 configured for insertion into bone and a head portion (not shown) defining a slot, opening or recess 214 for receiving a driving device. By way of example only, slot 214 may engage a screw driver or more specifically a hex screw driver (not shown). Pivotably mounted on base 210 is housing 220. Housing 220 includes an inner and outer housing 222, 224 and defines a rod receiving channel 223 therethrough. Inner and outer housings 222, 224 are configured to move relative to one another to selectively retain connector rod 50 (FIG. 1A).

For a more detailed description of the preferred low profile screw and the operation thereof, please refer to commonly owned U.S. Patent Application Publication No. 2007/0093817 and commonly owned U.S. patent application Ser. No. 11/493,625, the contents of which are hereby incorporated herein in their entirety. The preferred low profile screw is available under the brand name MESA® (K2M, Inc., Leesburg, Va.). However, other designs of low profile screws also are contemplated, such as the design shown and described in published U.S. Patent applications 2006/0276792 and 2007/0225711. As used herein, the term “low profile screw” contemplates any screw design which presents little or no profile above the rod. The preferred low profile spinal screw is the MESA® taper lock screw.

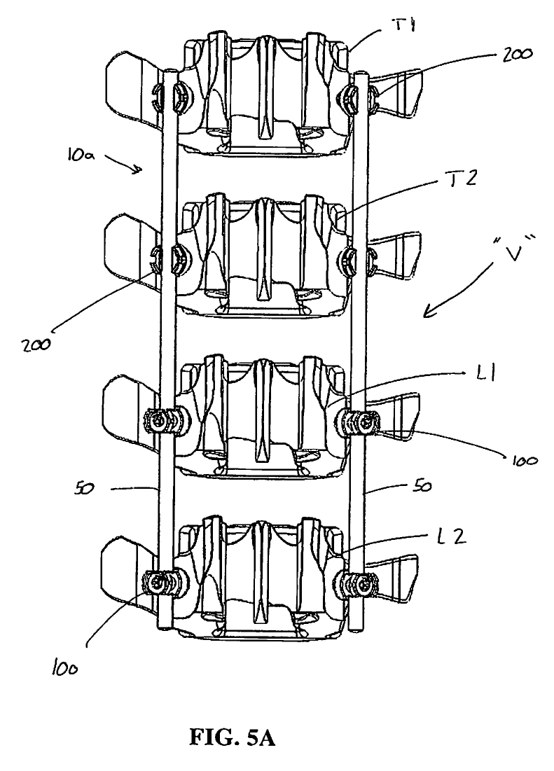

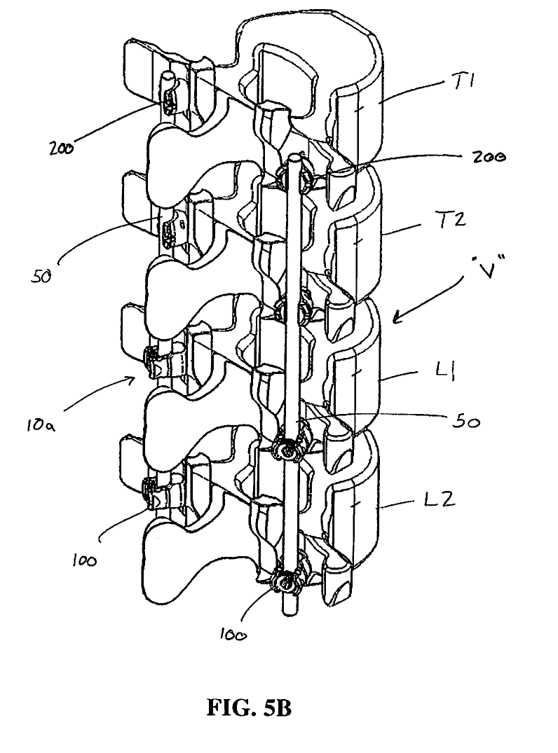

Turning now to FIGS. 5A and 5B, spinal fixation system 10a is shown securely affixed to a portion of a vertebral column “V”. Vertebral column “V” includes thoracic vertebra T1, T2 and lumbar vertebrae L1, L2. Although the aspects of the present disclosure may be utilized through the vertebral column, the following discussion will relate to the use of spinal fixation system 10 on thoracic vertebrae T1, T2 and lumbar vertebrae L1, L2.

Initially, bases 110 of set screw assemblies 100 are positioned and implanted within lumbar vertebra L1, L2 and bases 210 of low profile screw assemblies 200 are positioned and implanted within thoracic vertebra T1, T2. As discussed above, each of bases 110, 210 (FIG. 1A) include a slot 114, 214 (FIGS. 2B and 4B, respectively) for engaging a driving device. Once properly positioned and implanted within vertebral column “V”, as discussed in the above referenced documents, connecting rods 50 may be inserted within rod receiving channels 123, 223 (FIGS. 2A and 4A, respectively) of screw assemblies 100, 200, respectively. Coupling elements 120 of set screw assemblies 100 may be adjusted as necessary to permit the insertion of connector rods 50. Housings 220 of low profile screw assemblies 200 may also be adjusted as necessary to accommodate the receipt of connector rods 50. Set screw assemblies 100 and low profile screw assemblies 200 may be locked, unlocked, adjusted and relocked as necessary until proper alignment of vertebral column “V” is achieved.

As discussed above, low profile screw assembly 200 presents little to no profile above connector rod 50. This low profile is most important in the area of the spine where there is less soft tissue coverage, i.e. the thoracic region. The use of set screw assembly 100 in these regions may cause patient discomfort or undue scarring. Additionally, the thoracic region is where most deformity correction is required. The locking and partial locking features of low profile screw assembly 200 permits the adjustment of screw assembly 100 as a deformity is corrected. However, in a construct which extends from the thoracic region of the spine to the lumbar region of the spine, the rod usually curves in a posterior to anterior direction. As a result, there may be a perception that the forces on the rod after implantation might cause the rod to try to dislocate from a low profile screw, especially a low profile screw which appears open at the top thereof. Thus, it is believed that the combination of a low profile screw in the thoracic region, where its advantages may be more appreciated, with a set screw type locking pedicle screw in the lumbar region, may be perceived to provide advantages to the surgeon and patient. In addition, premium priced low profile screws may be perceived as unnecessary in portions of the construct where the advantages of such a structure are not realized, such as the lumbar region, and so a more economical approach can be achieved by utilizing low profile screws where warranted and set screw style pedicle screws where such screws will suffice.

Maintenance and removal of spinal fixation system 10a is the same as would be for traditional set screw assemblies 100 and low profile screw assemblies 200 individually. Each of traditional set screw assemblies 100 and low profile screw assemblies 200 may be adjusted as necessary until the desired spinal alignment is achieved.

It will be understood that various modifications may be made to the embodiments herein. For example, fixed angle screws, uniplanar screws, monoaxial rotating screws, hooks, wires and cables may be used in combination with traditional set screw assembly 100 and low profiles screw assembly 200.

The pedicle screws of the present construct may be fabricated from titanium, titanium alloys, stainless steel, nickel titanium, polyetheretherketone (PEEK) or any other suitable biocompatible material. The rods used in the present construct may be fabricated from the same or dissimilar materials to the screws. Thus, if the screws are fabricated from titanium, titanium alloy, stainless steel, cobalt chrome (CoCr) or PEEK, then so too can the rod be fabricated from the same material. On the other hand, the rod may also be fabricated from a dissimilar, compatible material. Thus, the rod may be fabricated from PEEK and used with screws fabricated from any of the foregoing materials. The rod also may be fabricated from CoCr with the screws fabricated from titanium, titanium alloy, cobalt chrome, stainless steel, nickel titanium, or PEEK.

What is claimed is:

1. A spinal fixation system comprising,
at least one low profile screw assembly;
at least one bone screw assembly that is not a low profile screw assembly; and
a rod operably connectable between said at least one low profile screw assembly and at least one bone screw assembly that is not a low profile screw assembly.

2. The system of claim 1, wherein the at least one low profile screw assembly is a taper lock screw.

3. The system of claim 1, wherein the at least one bone screw assembly that is not a low profile screw includes a locking insert.

4. The system of claim 1, wherein the at least one bone screw assembly that is not a low profile screw includes a locking nut.

5. The system of claim 1, wherein the at least one bone screw assembly that is not a low profile screw includes a set screw.

6. The system of claim 1, further including at least one laminar hook operably connected to the rod.

7. The system of claim 1, further including at least one connector connected to the rod.

8. The system of claim 1, wherein the low profile screw and assembly and the bone screw that is not a low profile screw are made of a titanium or a titanium alloy and the rod is made of cobalt chrome.

9. The system of claim 1, wherein the low profile screw and assembly and the bone screw that is not a low profile screw are made of a titanium or a titanium alloy and the rod is made of PEEK.

10. A method for immobilizing a spine, the method comprising the steps of:
implanting at least one low profile screw assembly into a first portion of the spine;
implanting at least one bone screw assembly that is not a low profile screw into a second portion of the spine; and
connecting a rod to both the at least one low profile screw assembly to the at least one bone screw assembly which is not a low profile screw.

11. The method of claim 10, wherein the at least one low profile screw assembly is implanted in a thoracic vertebra.

12. The method of claim 10, wherein the at least one screw which is not a low profile screw is implanted in a lumbar vertebra.

13. The method of claim 10, further including the step of locking the rod to the at least one low profile screw assembly.

14. The method of claim 10, further including the step of locking the rod to the at least one screw which is not a low profile screw.

15. The method of claim 10, further comprising partially locking said low profile screw to said rod.

16. The method of claim 15 further comprising manipulating the spine with the low profile screw in the partially locked position.

17. The method of claim 10 wherein the step of connecting a rod comprises connecting a rod made from cobalt chrome.

18. The method of claim 17 further comprising the step of connecting a cobalt chrome rod to screws made from titanium or a titanium alloy.
